(19) United States
(12) Patent Application Publication
(10) Pub. No.: US 2007/0078143 A1
(43) Pub. Date: Apr. 5, 2007

(54) Method for preparing Ziprasidone monohydrochloride-hydrate

(75) Inventors: Siddiqui Mohammed Jaweed Mukarram, Aurangabad (IN); Aravind Yekanathsa Merwade, Aurangabad (IN); Kumar Kamlesh Laxmi Singh, Aurangabad (IN); Pawan Vrajlal Solanki, Aurangabad (IN)

Correspondence Address: IPLM GROUP, P.A., POST OFFICE BOX 18455, MINNEAPOLIS, MN, 55418, US

(73) Assignee: Wockhardt Limited

(21) Appl. No.: 11/441,913

(22) Filed: May 26, 2006

(30) Foreign Application Priority Data
Nov. 28, 2003 (WO) PCT/IB03/05479

(51) Int. Cl. A61K 31/496; C07D 417/14

(52) U.S. Cl. 514/254.04; 544/368

(57) ABSTRACT

Ziprasidone is a known agent for treating various disorders including schizophrenia, migraine pain and anxiety. The present invention provides a method for preparing 5-(2-(4-1,2-benzisothiazol-3-yl)piperazinyl)ethyl)-6-chloro-1,3-dihydro-2H-indol-2-one (Ziprasidone) monohydrochloride-hydrate or a pharmaceutically acceptable acid addition salt thereof.

### CROSS-REFERENCE TO RELATED APPLICATION

This application claims priority from international patent application Serial No. PCT/IB2003/005479 filed Nov. 28, 2003, and published in English on Jun. 16, 2005 as International Publication No. WO 2005/054235 A1, which are incorporated herein by reference.

### FIELD OF THE INVENTION

The present invention relates to a method for the preparation of 5-(2-(4-1,2-benzisothiazol-3-yl)piperazinyl)ethyl)-6-chloro-1,3-dihydro-2H-indol-2-one (Ziprasidone) monohydrochloride-hydrate having Formula I 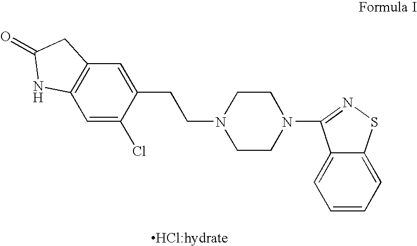
The compound of Formula I is prepared by condensing 1,2-benzisothiazole-3-piprazinyl hydrochloride with 2-chloroethyl-6-chlorooxindole in presence of sodium iodide. The crude product can be purified by re-crystallization in a mixture of tetrahydrofuran and dimethyl formamide. The free compound can be converted into a hydrochloride hydrate in aqueous methanol having characteristic IR, TGA and DSC properties.

### BACKGROUND OF THE INVENTION

The activity of 5-(2-(4-1,2-benzisothiazol-3-yl)piperazinyl)ethyl)-6-chloro-1,3-dihydro-2H-indol-2-one (Ziprasidone) and its pharmaceutically acceptable salts as a neuroleptic make these compounds useful for treating psychotic disorders in human subjects. For example, Ziprasidone is useful for treating psychotic disorders of the schizophrenic types. 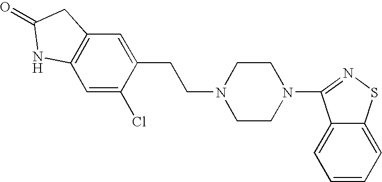
In particular, it is useful for removing or ameliorating such symptoms as anxiety, agitation, excessive aggression, tension and social or emotional withdrawal in psychotic patients. Ziprasidone is a known compound having the chemical structure:

Arylpiperazinylethyl heterocyclic compounds and their use in the treatment of psychiatric disorders are disclosed in the U.S. Pat. No. 4,558,060 (“the '060 patent”). The aryl group in the '060 patent is pyrimidinyl or an optionally substituted phenyl. Compounds with a butyl group between the piperazinyl and heterocyclic group are not disclosed, and heterocyclic groups other than benzoxazolones are not disclosed.

U.S. Pat. No. 4,831,031 discloses that arylpiperazinyl ethyl (or butyl) heterocyclic compounds can be prepared by using a reaction of an arylpiperazine having Formula 1 with a fused bicyclic compound having Formula 2 as follows: 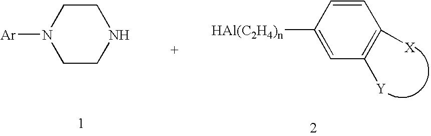
The coupling reaction is generally conducted in a polar solvent (such as a lower alcohol, dimethylformamide or methylisobutyl ketone) and in the presence of a weak base and preferably the reaction is carried out in the presence of a catalytic amount of sodium iodide and a neutralizing agent for hydrochloride such as sodium carbonate.

Yevich, et al, J. Med. Chem, 29, No.3, page 359 to 369 (1986) discloses a method of producing 1-(1,2-benzisothiazol-3-yl) and (1,2-benzisoxazole-3-yl)piperazine derivatives. Several reaction schemes are disclosed including reaction schemes wherein coupling occurs in a free base.

U.S. Pat. No. 5,359,068 discloses the synthesis of Ziprasidone by treating a compound having the formula: 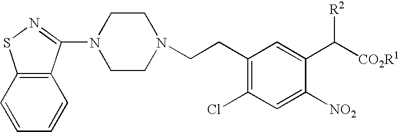
wherein R² is hydrogen, CN, or CO₂R¹, and R¹ is hydrogen or (C₁₋₆) alkyl; with a reducing agent selected from the group consisting of sodium hydrosulfite, hydrogen in the presence of a hydrogenation catalyst, iron in acetic acid, zinc and calcium chloride in acetic acid and NH₂—PO₂ in the presence of Pd/C with the proviso that when R² is CN or CO₂R¹ or (C₁₋₆) alkyl the product of the reduction is heated with an 6 or 3N hydrochloric acid or acetic acid.

U.S. Pat. No. 6,150,366 discloses a manufacturing process for Ziprasidone hydrochloride monohydrate, which includes:
(a) dissolving Ziprasidone free base in a solvent comprising THF and water, in a volume ratio of about 22-35 unit volumes of THF to about 1.5-8 volumes of water;
(b) heating the solution resulting from step (1);
(c) adding HCl to the solution resulting from step (2); and
(d) cooling the solution resulting from step (3) and crystals were collected by filtration and drying.

U.S. Pat. No. 5,312,925 (“the '925 patent”) discloses a process for the synthesis of monohydrate of 5-(2-(4-1,2-benzisothiazol-3-yl)piperazinyl)ethyl)-6-chloro-1,3-dihydro-2H-indol-2-one hydrochloride. The compound is characterized based on IR, XRD and moisture content. The '925 patent also discloses that the hemihydrate may be obtained by the process disclosed in Example 16 of U.S. Pat. No. 4,831,031 and characterized by IR, XRD and moisture content. It also discloses the IR, XRD and moisture content of anhydrous Ziprasidone hydrochloride. According to the '925 patent specification samples having water content of 3.97, 2.55 and 0.37% were used for the IR and XRD study of Ziprasidone hydrochloride monohydrate, hemihydrate and anhydrous.

There is a need for a method to prepare stable crystalline Ziprasidone hydrochloride hydrate.

### SUMMARY OF THE INVENTION

The present invention provides a method for manufacturing 5-(2-(4-1,2-benzisothiazol-3-yl)piperazinyl)ethyl)-6-chloro-1,3-dihydro-2H-indol-2-one monohydrochloride-hydrate, which has utility as a neuroleptic agent, and is thus useful as an antipsychotic agent.

In a first embodiment, the invention is directed to crystalline Ziprasidone hydrochloride hydrate.

In a second embodiment, the invention is directed to a method for the preparation of a compound having Formula I, comprising reactions of piperazine hydrochloride salt derivatives of Formula II with alkyl halide derivatives of oxoindole of Formula III: 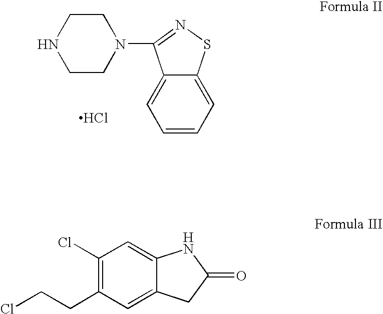
in aqueous medium using NaI as catalyst. The yield of the desired product is high and purity is more than 99.5% (based on HPLC assessment). The moisture content in the Formula I product is around 5.0 to 6.0% by weight. This is in contrast to the product disclosed in U.S. Pat. No. 5,312,925 which has water content of 3.97, 2.55 and 0.37%. The Ziprasidone salt of the present invention has the water content as surface water instead of water of crystallization and this is supported by IR Spectrum, Thermal Gravimetric Analysis (TGA) and Differential Scanning Calorimetry (DSC). The hydrated form of Ziprasidone hydrochloride in the present invention is neither monohydrate nor hemihydrate as evident from their IR spectrum and TGA analysis. TGA clearly demonstrates that water content (up to 6%) which is associated with the sample of this invention is not the water of crystallization, since water loss occurs at 85 to 90° C.

### BRIEF DESCRIPTION OF THE DRAWINGS

FIG. 1 is an Infra Red Spectrum of Ziprasidone hydrochloride hydrate of the invention.

FIG. 2 is a Thermal Gravimetric Analysis of Ziprasidone hydrochloride hydrate of the invention.

FIG. 3 is a Differential Scanning Calorimetry (DSC) of Ziprasidone hydrochloride hydrate of the invention.

### DETAILED DESCRIPTION OF THE INVENTION

The present invention is directed to an anti-psychiatric disorder compound, Ziprasidone, having therapeutic value and a method for manufacturing the monohydrochloride-hydrate of Ziprasidone. Ziprasidone has utility as a neuroleptic, and is thus is useful as an antipsychotic. In particular, the present invention is directed to, Ziprasidone, the monohydrochloride salt of 5-(2-(4-1,2-benzisothiazol-3-yl)piperazinyl)ethyl)-6-chloro-1,3-dihydro-2H-indol-2-one hydrate.

In a first embodiment, the invention is directed to crystalline Ziprasidone hydrochloride hydrate having the chemical Formula I: 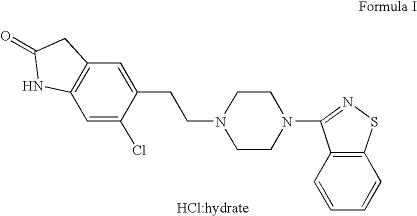
and a method for manufacture the compound of Formula I. The monohydrate of Ziprasidone hydrochloride is prepared by reacting anhydrous 5-(2-(4-1,2-benzisothiazol-3-yl)piperazinyl)ethyl)-6-chloro-1,3-dihydro-2H-indol-2-one with aqueous hydrochloric acid. The temperature range of the reaction was maintained at about 60 to 65° C. and aqueous HCl used for salt formation was around 0.7 M. Depending on the reaction temperature and other conditions, the reaction times are about 3 to 24 hours. The final product is dried under carefully monitored conditions to make certain that water content was from about 3.8% to about 4.5%, to obtain the stable monohydrate.

According to one embodiment the present invention, 5-(2-(4-1,2-benzisothiazol-3-yl)piperazinyl)ethyl)-6-chloro-1,3-dihydro-2H-indol-2-one and the hydrated hydrochloride salt are prepared according to the following synthetic reaction scheme:

(a) Reaction of 1,2-benzisothiazole-3-piperazinyl HCl with 2-Chloroethyl-6-chloro Oxoindole

1,2-Benzisothiazole-3-piperazinyl hydrochloride (Formula II): 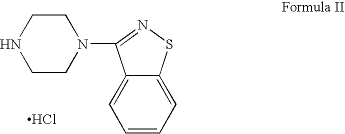
is reacted with a molar excess of 2-Chloroethyl-6-chloro oxoindole (Formula III) in a solvent and in the presence of a mild base to form 5-(2-(4-1,2-benzisothiazol-3-yl)piperazinyl)-ethyl)-6-chloro-1,3-dihydro-2H-indol-2-one (Formula IV).

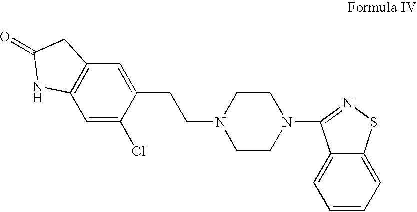

The molar excess of 2-Chloroethyl-6-chloro oxoindole used in this reaction stage is typically between about 1 to about 3 fold, and preferably about 2 fold. Suitable solvents for this synthetic stage include water. 1,-Benzisothiazole-3-piprazinyl hydrochloride is added to the aqueous sodium carbonate solution at ambient temperature. (The sodium carbonate is dissolved in water to form a 2-6% solution by weight.) The mixture is stirred for one hour to obtain a suspension. 2-Chloroethyl-6-chloro-oxindole, followed by sodium iodide is added to the suspension and the combined mixture is heated at a temperature of from about 50 to 100° C. for 20 to 30 hours. After completion of the reaction, the mixture was cooled to ambient temperature and filtered. The crude Ziprasidone obtained is again heated with water, cooled to room temperature, filtered and dried to obtain in the purified product in greater than 95% HPLC purity.

In the present invention the coupling reaction is conducted in water. This in-situ aqueous based coupling process is more efficient and has a lower environmental burden because the handling and disposal of organic solvents are eliminated. This process has not shown formation of any side products and does not require special isolation procedures process, e.g., extraction or distillation.

(b) Purification of Crude Ziprasidone

Ziprasidone has poor solubility in most of the common organic solvents. Therefore, another objective of the present invention is to find a suitable solvent or solvent mixture to purify crude Ziprasidone base to obtain the required purity with minimal loss of material. The crude Ziprasidone obtained from step (a) is dissolved in a mixture of tetrahydrofuran and N,N-dimethylformamide (7:3, v/v) with activated charcoal and heated to clarify the solution. The solution obtained is filtered, washed with a cold solvent mixture of THF and DMF and dried. The dried Ziprasidone is obtained in 99% HPLC purity.

(c) Reaction of 5-(2-(4-1,2-benzisothiazol-3-yl)piperazinyl)ethyl)-6-chloro-1,3-dihydro-2H-indol-2-one with Hydrogen Chloride

5-(2-(4-1,2-Benzisothiazol-3-yl)piperazinyl)ethyl)-6-chloro-1,3-dihydro-2H-indol-2-one is reacted with hydrogen chloride in aqueous organic solvent, to provide 5-(2-(4-1,2-benzisothiazol-3-yl)piperazinyl)ethyl)-6-chloro-1,3-dihydro-2H-indol-2-one hydrochloride hydrate (Formula I).

Suitable aqueous organic solvents for the hydrochlorination reaction include, but are not limited to, organic solvents such as aliphatic alcohols, e.g., methanol, ethanol, 2-propanol or n-butanol, and the like. The volume ratio of alcohol and water is typically from about 1:1 to about 8:2 more particularly about 7:3. The hydrochloric acid is added to the solution drop-wise and stirred for about 20 hours at about 65° C. The amount of hydrochloric acid solution in the reaction is from about 5.0 to about 7.0% by weight or about 6.0 moles of concentrated HCl.

After washing with the cold solvent mixture of THF and DMF solvents, Ziprasidone is dried at 60° C. for 10 to 12 hours to obtain the 5-(2-(4-1,2-benzisothiazol-3-yl)piperazinyl)ethyl)-6-chloro-1,3-dihydro-2H-indol-2-one hydrochloride hydrate (Formula I). The final product is 99.5% pure based on HPLC and has the characteristic IR, TGA and DSC values which confirm the product is Ziprasidone monohydrochloride-hydrate. The Ziprasidone monohydrochloride salt has a moisture content in the range of 5.0% to 6.0%. The Ziprasidone salt of the present invention has the water as surface water instead of water of crystallization. This is confirmed by the IR Spectrum, Thermal Gravimetric Analysis (TGA) and Differential Scanning Calorimetry (DSC).

Anhydrous Ziprasidone monohydrochloride is disclosed in U.S. Pat. No. 5,312,925. The Ziprasidone monohydrochloride-hydrate of the present invention can be made anhydrous having moisture content up to 0.7% by weight, after prolonged drying. It has been observed that the compound re-absorbs moisture up to 6% when it is exposed at 60% relative humidity.

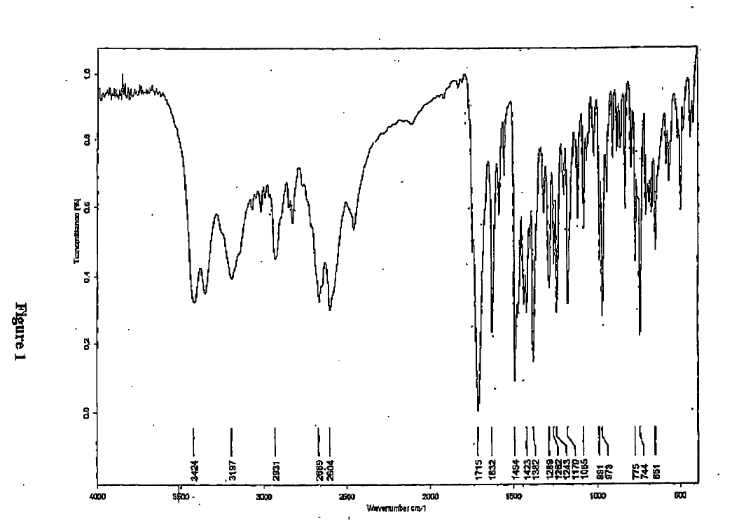

The IR spectrum of Ziprasidone of the present invention is characterized by the following peaks at about 3424, 3197, 2931, 2669, 2604, 2458, 1715, 1632, 1494, 1382, 1289, 1262, 1243, 1179, 1085, 991, 973, 775, 744 and 651 cm⁻¹ (FIG. 1). The infrared spectrum of Ziprasidone monohydrochloride-hydrate shows sharp bands at 3424 cm⁻¹ and 2458 cm⁻¹ in contrast to the IR spectrum of the Ziprasidone monohydrochloride monohydrate disclosed in the U.S. Pat. No. 5,312,925.

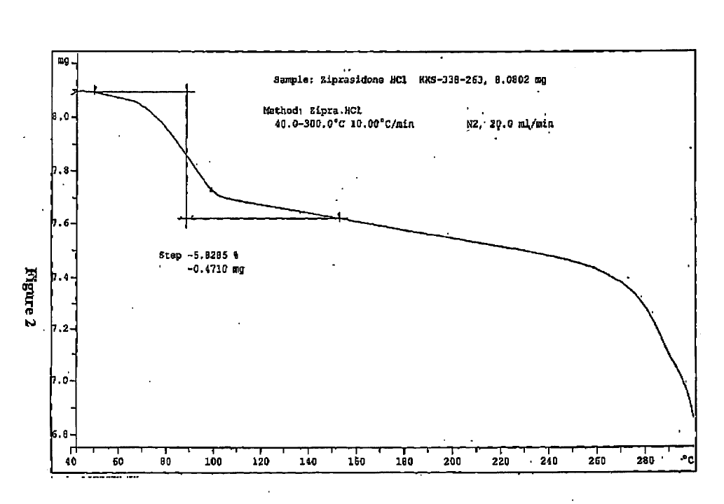

Thermal Gravimetric Analysis (TGA) of the crystalline Ziprasidone hydrochloride hydrate of the present invention is characterized by the weight loss at about 85 to 90° C. (FIG. 2). The TGA data of Ziprasidone monohydrochloride-hydrate showed a weight loss at 85 to 90° C. indicating that the moisture lost is surface water and not the water of crystallization.

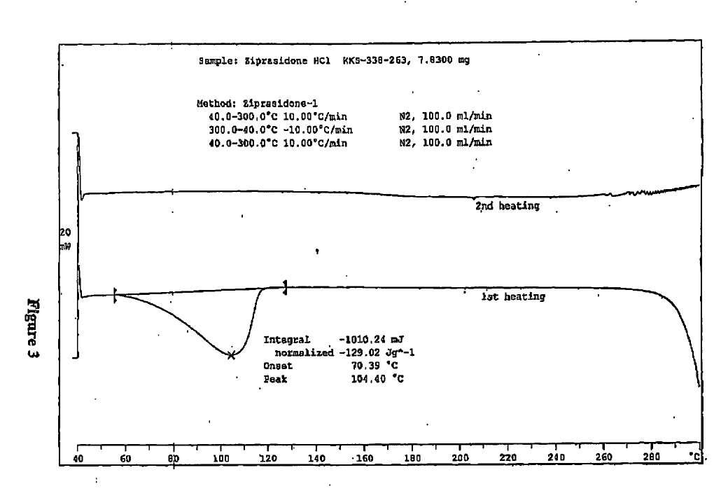

The Differential Scanning Calorimetry (DSC) thermogram of 5-(2-(4-1,2-benzisothiazol-3-yl)piperazinyl)ethyl)-6-chloro-1,3-dihydro-2H-indol-2-one hydrochloride hydrate of the invention shows a peak endotherm at 104.40° C. (FIG. 3).

Based on the above data from the IR and TGA, the product of the present invention, Ziprasidone Monohydrochloride, has only surface moisture and not as water of crystallization.

The following examples illustrate the invention, but is not limiting thereof.

### EXAMPLE 1

### 5-(2-(4-(1,2-benzisothiazol-3-yl)-1-piperazinyl)ethyl)-6-chloro-1,3-dihydro-2H-indole-2-one (Ziprasidone)

A 10 liter three-necked round bottom flask is charged with sodium carbonate (742 g) and pure water (3.06 liter). 1,2-Benzisothiazole-3-piperazinyl HCl (611 g, 2 moles) is added to the sodium carbonate solution at ambient temperature and the reaction mixture stirred for an additional 1 hour. To the suspension 2-chloroethyl-6-chloro oxoindole (430 g, 2 moles) and sodium iodide (20 g) is added. The combined reaction mixture is heated at 60 to 90° C. for 20 to 30 hours. After completion of the reaction, the reaction mixture is cooled to ambient temperature and the slurry obtained is stirred for 1 additional hour and filtered. The wet tan colored crude free base is suspended in water (7.50 liter), heated to 75 to 80° C. with stirring for 1 hour and filtered at a temperature of 45 to 50° C. The wet compound is dried at 65 to 70° C. for 10 to 12 hours to obtain 752 gm of the desired product (HPLC Purity 95 to 96%).

### EXAMPLE 2

### Crystallization of Crude Ziprasidone Base

A mixture of tetrahydrofuran:dimethylformamide (30 liter, 7:3, v/v), and crude Ziprasidone (750 gm) is charged with activated charcoal (15 gm) and stirred for 1 hour at 65 to 67° C. The clear solution is filtered under vacuum and solution is concentrated up to a volume of 3.0 to 5.0 liters. The slurry obtained is cooled to a temperature of 0 to 5° C. and stirred for an additional 1 hour. The crystallized product is filtered and washed with a mixture of tetrahydrofuran:dimethylformamide (250 ml), and dried at 60 to 70° C. under high vacuum for 10 to 12 hours to obtain 5-(2-(4-1,2-benzisothiazol-3-yl)piperazinyl)ethyl)-6-chloro-1,3-dihydro-2H-indol-2-one free base (603 g) in greater than 99% HPLC purity.

### EXAMPLE 3

### 5-(2-(4-1,2-benzisothiazol-3-yl)piperazinyl)ethyl)-6-chloro-1,3-dihydro-2H-indol-2-one Monohydrochloride-hydrate

A 10 liter three necked round bottom flask is charged with 5-(2-(4-(1,2-benzisothiazol-3-yl)-1-piperazinyl)-ethyl)-6-chloro-1,3-dihydro-2H-indol-2-one free base (600 g, 6.452 mole) and a mixture of methanol:water (6 liter, 7:3 v/v). The slurry is stirred and concentrated hydrochloride acid (900 g) is added through a dropping funnel over 30 minutes. The slurry is allowed to stand at 65° C. for 20 hours. The reaction mixture is cooled to room temperature, filtered, washed with 500 ml of cold methanol:water mixture and dried at 60° C. for 10 to 12 hours. The reaction provides 5-(2-(4-1,2-benzisothiazol-3-yl)piperazinyl)ethyl)-6-chloro-1,3-dihydro-2H-indol-2-one monohydrochloride-hydrate (665 g) in 99.5% HPLC purity.

All patents, patent applications, and literature cited in the specification are hereby incorporated by reference in their entirety. In the case of any inconsistencies, the present disclosure, including any definitions therein will prevail.

We claim:

1. A method for preparing a monohydrochloride-hydrate compound of Formula I: 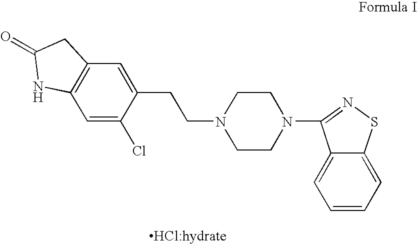
comprising; (a) contacting a compound having Formula IV 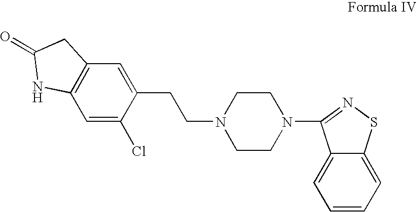
with hydrochloric acid in the mixture of solvents for a sufficient time to form 5-(2-(4-1,2-benzisothiazol-3-yl)piperazinyl)ethyl)-6-chloro-1,3-dihydro-2H-indol-2-one monohydrochloride-hydrate; and
(b) isolating the 5-(2-(4-1,2-benzisothiazol-3-yl)piperazinyl)ethyl)-6-chloro-1,3-dihydro-2H-indol-2-one monohydrochloride-hydrate.

2. The method of claim 1, wherein the hydrochlorination step (a) is carried out in aqueous organic solvent.

3. The method of claim 2, wherein the organic solvent is an aliphatic alcohol.

4. The method of claim 3, wherein the aliphatic alcohol is methanol, ethanol, 2-propanol, or n-butanol.

5. The method of claim 4, wherein more particularly aliphatic alcohol is methanol.

6. The method of claim 5, wherein the methanol and water have a volume ratio of about 1:1 to about 8:2.

7. The method of claim 6, wherein the methanol and water have a volume ratio of about 7:3.

8. The method of claim 1, wherein 5-(2-(4-1,2-benzisothiazol-3-yl)piperazinyl)ethyl)-6-chloro-1,3-dihydro-2H-indol-2-one is charged to methanol-water mixture prior to addition of hydrochloric acid.

9. The method of claim 8, wherein the hydrochloric acid is concentrated.

10. The method of claim 1, wherein the 5-(2-(4-1,2-benzisothiazol-3-yl)piperazinyl)-ethyl)-6-chloro-1,3-dihydro-2H-indol-2-one and hydrochloric acid have a molar contact ratio of about 1:1 to about 1:2 mol/g.

11. The method of claim 10, wherein the molar ratio is about 1:1.5 mol/g.

12. The method of claim 1, wherein the contact time is from about 15 to about 25 hours.

13. The method of claim 1, wherein contacting step (a) is carried out at about 60° C. to about 70° C.

14. The method of claim 1, wherein isolating comprises cooling the mixture to room temperature and filtering the product.

15. The method of claim 14, wherein isolating further comprises drying the product at a temperature of about 55° C. to 65° C.

16. The method of claim 15, wherein drying time is between about 10 to about 12 hours.

17. The method of claim 1, wherein the compound of Formula I is isolated in more than about 99.4% purity.

18. A monohydrochloride-hydrate compound of Formula I: 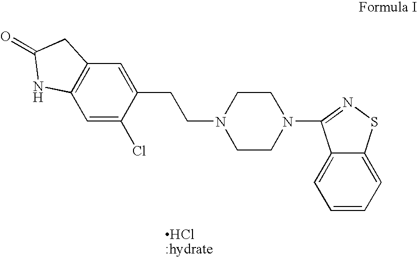
or a pharmaceutically acceptable acid addition salt thereof.

19. The compound of claim 18, wherein the 5-(2-(4-1,2-benzisothiazol-3-yl)piperazinyl)ethyl)-6-chloro-1,3-dihydro-2H-indol-2-one monohydrochloride-hydrate is characterized by an Infra Red Spectroscopy (IR) spectrum having peaks at about 3424, 3197, 2931, 2669, 2604, 2458, 1715, 1632, 1494, 1382, 1289, 1262, 1243, 1179, 1085, 991, 973, 775, 744 and 651 cm⁻¹.

20. The compound of claim 18, wherein TGA of the 5-(2-(4-1,2-benzisothiazol-3-yl)piperazinyl)ethyl)-6-chloro-1,3-dihydro-2H-indol-2-one monohydrochloride-hydrate is characterized by the weight loss from about 85 to 90° C.

21. The method of claim 20, wherein weight loss in TGA at about 85 to 90° C. indicates the moisture content is surface moisture.

22. The compound of claim 18, wherein DSC of the 5-(2-(4-1,2-benzisothiazol-3-yl)-piperazinyl)ethyl)-6-chloro-1,3-dihydro-2H-indol-2-one monohydrochloride-hydrate is characterized by a peak endotherm at about 104.40° C.
